(19) United States
(12) Patent Application Publication
(10) Pub. No.: US 2024/0404646 A1
(43) Pub. Date: Dec. 5, 2024

(54) Ms/Ms-Based Identification of Trisulfide Bonds

(71) Applicant: DH Technologies Development Pte. Ltd., Singapore (SG)

(72) Inventor: Yuzhuo ZHANG, Redwood City, CA (US)

(73) Assignee: DH Technologies Development Pte. Ltd., Singapore (SG)

(21) Appl. No.: 18/694,233

(22) PCT Filed: Sep. 21, 2022
(86) PCT No.: PCT/IB2022/058925

§ 371 Date: Mar. 21, 2024

Related U.S. Application Data
(60) Provisional application No. 63/246,878, filed on Sep. 22, 2021.

(51) Int. Cl. G16C 20/20; G01N 33/68; G16B 40/10; H01J 49/00; H01J 49/42

(52) U.S. Cl. CPC G16C 20/20; G01N 33/6848; G01N 33/6854; G16B 40/10; H01J 49/0036; H01J 49/005; H01J 49/0054; H01J 49/421

(57) ABSTRACT

Methods and system that determines disulfide and trisulfide linkages within analytes (e.g., polypeptides) is described. In certain aspects. a sample comprising polypeptides (such as an antibody) may be subjected to dissociation using an electron activated dissociation (which can include electron capture dissociation and electron transfer dissociation) and the fragmentated portions are analyzed using a mass spectrometer to produce a spectrum. The spectrum is analyzed by a processor to identify peaks from the spectrum that are related to one another in the spectra by a separation of 32 mass units. In identifying an antibody comprising peptide segments linked via a trisulfide bond. for example, four different peaks representing two different peptides are searched for and identified representing a first peptide portion having mass/charge of A and A+32 and a second peptide having mass/charge of B and B+32.

### FIELD

The teachings herein relate to methods and systems of analyzing compounds using mass spectrometry, and more particularly, methods and systems for determining the presence of trisulfide bonds using MS/MS-based analysis.

### BACKGROUND

Mass spectrometry (MS) is an analytical technique for measuring the mass-to-charge ratios (m/z) of molecules within a sample, with both quantitative and qualitative applications. For example, mass spectrometry can be used to identify unknown compounds in a test substance, determine the isotopic composition of elements in a specific molecule, determine the structure of a particular compound by observing its fragmentation, and/or quantify the amount of a particular compound in a test sample. MS typically involves converting the sample molecules into ions using an ion source and separating and detecting the ionized molecules with electric and/or magnetic fields due to differences in their mass-to-charge ratios (m/z) using one or more mass analyzers. Depending on the experiment, ions generated by the ion source may be detected intact (generally referred to as MS) or alternatively may be subject to fragmentation as in tandem MS (also referred to as MS/MS or MS2) such that product ions resulting from the fragmentation of selected precursor ions may additionally or alternatively be detected.

Excessive and/or unintended fragmentation of analytes of interest may render the interpretation of MS/MS data difficult. By way of example, fragmentation of multiply-charged precursor ions may generate a large number of highly-charged fragments, which themselves may further fragment, thereby adding undesired noise to the MS/MS data rendering reconstruction of the analyte structure difficult. In MS-based proteomics, for example, such fragmentation may conceal abnormal amino acid sequences and/or important post-translational modifications. One such example of a post-translational modification of interest is the insertion of a sulfur atom into a disulfide bond to form a trisulfide linkage, which has been observed in both natural and recombinant antibodies.

There remains a need for reliable techniques for generating and interpreting MS/MS data to identify trisulfide bonds.

### SUMMARY

In accordance with the present teachings, methods, systems and devices are disclosed for determining the presence of trisulfide bonds within peptides using MS/MS-based analysis.

In one aspect of the present teachings, a computer-implemented method of determining the presence of trisulfide bonds in a sample is provided, the method comprising instructing, using a processor, a fragmentation device to generate a plurality of fragment ions from a population of analyte ions and instructing, using the processor, a mass analyzer to generate data indicative of the m/z of the plurality of fragment ions. Based on the data indicative of the m/z of the plurality of fragment ions, the computer-implemented method can identify at least a first pair of fragment ions, if any, differing in mass from one another by about 32 mass units using the processor.

The fragment ions can be generated in a variety of manners such as collision induced dissociation (CID) or electron activated dissociation (EAD). In certain aspects, the fragmentation device may be configured to generate the plurality of fragment ions using an EAD technique. For example, the plurality of fragment ions may be generated by electron capture dissociation (ECD).

In addition to identifying a first pair of ions differing by 32 mass units from one another, the method may further comprise identifying a second pair of fragment ions differing in mass from one another by about 32 mass units using the processor, wherein the second pair of fragment ions differ in mass from the first pair of fragment ions.

In various aspects, the computer implemented method may further comprise instructing, using the processor, the mass analyzer to generate data indicative of the m/z of the population of analyte ions. In certain related aspects, the method may further comprise identifying a precursor ion corresponding to the first pair of fragment ions and a second pair of fragment ions identified based on the data indicative of the m/z of the population of analyte ions, wherein the second pair of fragment ions differ in mass from one another by about 32 mass units and wherein the second pair of fragment ions differ in mass from the first pair of fragment ions.

In various aspects, the sample may be subjected to liquid chromatography prior to being subject to fragmentation.

In various aspects, the method may further comprise generating a mass spectrum, using the processor, based on the measured m/z of the plurality of fragment ions and, using the processor, identifying at least two pairs of peaks in the mass spectrum, each pair corresponding to fragment ions differing in mass from one another by about 32 mass units.

The population of analyte ions can be various molecules. In certain aspects, the population of analyte ions may comprise polypeptides. In some related aspects, the polypeptides may comprise antibodies.

In accordance with various aspects of the present teachings, a method of determining the presence of trisulfide bonds in a sample is provided, the method comprising performing electron activated dissociation on a population of analyte ions to generate a plurality of fragment ions; and identifying two pairs of fragment ions from the plurality of fragment ions, wherein fragment ions in each pair differ in mass from one another by about 32 mass units.

In some related aspects, performing EAD may comprise performing ECD.

In some related aspects, the method may further comprise mass analyzing the plurality of fragment ions to measure the m/z and intensity of the plurality of fragment ions.

Additionally, in certain aspects, the method may further comprise mass analyzing the population of analyte ions to identify a precursor ion to the two pairs of fragment ions.

In some aspects, the sample may be subjected to liquid chromatography prior to being subjected to EAD.

The population of analyte ions can be various molecules. In certain aspects, the population of analyte ions may comprise polypeptides. In some related aspects, the polypeptides may comprise antibodies.

In accordance with various aspects of the present teachings, non-transitory machine readable storage medium storing one or more sequences of instructions executable by one or more processors to perform a set of operations for analyzing a sample is provided. In some aspects, the non-transitory machine readable storage medium may provide instructions executable by the one or more processors to perform for a set of operations comprising: instructing a fragmentation device to fragment said sample into a plurality of fragments; instructing a mass analyzer to analyze said plurality of fragments; receiving from said mass analyzer, data indicative of m/z of said plurality of fragments; identifying from said m/z data, any pairs of fragments ions differing in mass from one another by 32 mass units; and displaying to a user that the sample contains a trisulfide linkage. For example, the medium may comprise instructions for displaying to the user that two pairs of fragments are coupled via the trisulfide linkage.

In certain aspects, the medium may further comprise instructions for instructing the mass analyzer to analyze precursor ions within said sample; receiving from said mass analyzer, data indicative of m/z of said plurality of precursor ions; and identifying from said data indicative of the m/z of said plurality of precursor ions, one or more precursor ions formed from said pairs of fragments ions.

In accordance with various aspects of the present teachings, a computer implemented method for determining the presence of a trisulfide in a sample comprising: performing an electron activated dissociation on said sample to create a plurality of fragments and generating a mass spectrum by analyzing said plurality of fragments in a mass spectrometer. Two peaks in said mass spectrum can be identified that are spaced apart in said mass spectrum from one another by 32 mass units.

In certain aspects, the electron activated dissociation can be a result of electron capture dissociation.

In various aspects, said sample can be subjected to liquid chromatography prior to performing said electron activated dissociation.

In accordance with various aspects, the present teachings provide a non-transitory machine readable storage medium storing one or more sequences of instructions executable by one or more processors to perform a set of operations for analyzing a sample, the set of operations comprising: instructing an electron activated dissociation device to fragment said sample into a plurality of fragments, instructing a mass spectrometer to analyze said plurality of fragments, receiving from said mass spectrometer, data indicative of mass/charge ratios of said plurality of fragments, identifying from said mass spectrometer data, a pair of fragments that are spaced apart in mass/charge ratios by 32 units, and if said pair of fragments are identified, displaying to a user that the sample has a trisulfide linkage.

In certain related aspects, wherein two different pairs of fragments are identified wherein each fragment of each pair is spaced apart from the other fragment of the each pair by a mass/charge ratio of 32 units.

In certain aspects, the set of operations may further comprise: instructing a mass spectrometer to analyze said sample, receiving from said mass spectrometer data indicative of mass/charge ratios of said sample, and determining if a mass/charge ratio present in said data indicative of mass/charge ratios of said sample correlates to a molecule having the two different pairs of fragments.

In accordance with various aspects of the present teachings, a system for analyzing a sample is provided, the system comprising a tandem mass spectrometer and a processor. In certain aspects, the tandem mass spectrometer may comprise an ion filter, at least one of a collision cell and an electron activated dissociation device, and a mass analyzer. The processor may be configured to instruct the tandem mass spectrometer to perform a MS1 scan of the sample by mass analyzing the sample, receive from the tandem mass spectrometer an MS1 spectra of the sample, and identify at least one pair of peaks in the MS1 spectra that differ from one another in m/z by 32 units. For each of said at least one pair of peaks, the processor may be configured to instruct the tandem mass spectrometer to isolate a precursor ion representative of each of the peaks in each pair of the at least one pair of peaks and perform an electron activated fragmentation on each precursor ion and to generate fragment ions and to mass analyze said fragment ions, receive from the tandem mass spectrometer an MSMS spectra of said fragment ions, survey the MSMS spectra for one or more fragment peaks pairs, wherein in each fragment peak pair, the m/z for each differs from the other by 32, and determine whether the fragment peak pairs correlates by determining whether said fragment peaks were derived from the precursor ion of any of the at least one pair of peaks in the MS1 spectra and if so, determining that a tri-sulfide linkage exists in said sample.

These and other features of the applicant's teachings are set forth herein.

### BRIEF DESCRIPTION OF THE DRAWINGS

The skilled person in the art will understand that the drawings, described below, are for illustration purposes only. The drawings are not intended to limit the scope of the applicant's teachings in any way.

FIG. 1 schematically depicts the fragmentation of an exemplary analyte comprising a trisulfide linkage.

FIG. 2 depicts a flow chart of an example method of analyzing a sample in accordance with an aspect of various embodiments of the applicant's teachings.

FIG. 3 is a schematic representation of an exemplary mass spectrometer system in accordance with an aspect of various embodiments of the applicant's teachings.

FIG. 4 is a block diagram that illustrates a computer system, upon which embodiments of the present teachings may be implemented in accordance with various aspects of the applicant's teachings.

### DETAILED DESCRIPTION

It will be appreciated that for clarity, the following discussion will explicate various aspects of embodiments of the applicant's teachings, while omitting certain specific details wherever convenient or appropriate to do so. For example, discussion of like or analogous features in alternative embodiments may be somewhat abbreviated. Well-known ideas or concepts may also for brevity not be discussed in any great detail. The skilled person will recognize that some embodiments of the applicant's teachings may not require certain of the specifically described details in every implementation, which are set forth herein only to provide a thorough understanding of the embodiments. Similarly, it will be apparent that the described embodiments may be susceptible to alteration or variation according to common general knowledge without departing from the scope of the disclosure. The following detailed description of embodiments is not to be regarded as limiting the scope of the applicant's teachings in any manner.

As used herein, the terms “about” and “substantially equal” refer to variations in a numerical quantity that can occur, for example, through measuring or handling procedures in the real world; through inadvertent error in these procedures; through differences in the manufacture, source, or purity of compositions or reagents; and the like. Typically, the terms “about” and “substantially” as used herein mean 10% greater or lesser than the value or range of values stated or the complete condition or state. For instance, a concentration value of about 30% or substantially equal to 30% can mean a concentration between 27% and 33%. The terms also refer to variations that would be recognized by one skilled in the art as being equivalent so long as such variations do not encompass known values practiced by the prior art.

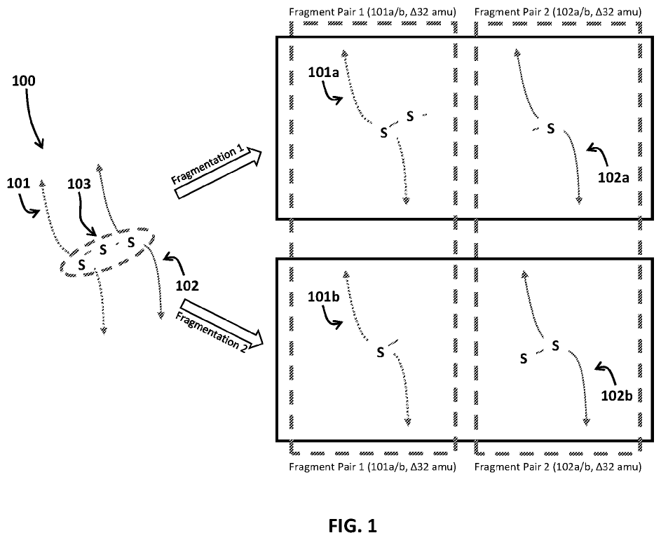

Systems and methods in accordance with various aspects of the present teachings enable the determination of trisulfide linkages within a population of analytes ions based on the MS/MS-based detection of one or more pairs of fragment ions differing from one another by about 32 mass units. While the fragment ions can be generated in a variety of manners such as collision induced dissociation (CID) or electron activated dissociation (EAD), it has been found that EAD techniques such as electron capture dissociation (ECD) may be particularly effective at fragmenting the precursor ions at the trisulfide bond such that for each trisulfide linkage, two pairs of fragment ions are reliably generated from the dissociation. With reference now to FIG. 1, a single exemplary analyte ion 100 is schematically depicted having a trisulfide linkage 103 linking a first portion 101 and a second portion 102 of the analyte ion 100. Upon being subjected to fragmentation conditions (e.g., as in CID or EAD) in accordance with various aspects of the present teachings, the trisulfide linkages 103 within a population of the analyte ions 100 can be broken such that the analyte ion 100 dissociates into first and second portions. In particular, as schematically depicted in the top solid box, a portion of the population of analyte ions 100 may be fragmented on one side of the central sulfur in the trisulfide bond such that first fragment 101a contains the central sulfur, while the second fragment 102a contains only one sulfur of the trisulfide bond 102. Alternatively, as schematically depicted in the top solid box, fragmentation of another analyte ion 100 among the population may result in dissociation on the other side of the central sulfur such that first fragment 101b contains only one sulfur of the trisulfide bond 102 while the second fragment 102b contains the central sulfur. Subsequent mass spectrometric detection of the various ion fragments generated from fragmentation of the population of analyte ions 100 would indicate the presence of at least four ions 101a, 102a, 101b, and 102b. As discussed otherwise herein, systems and methods in accordance with the present teaching may be configured to automatically determine (e.g., without user interaction) from the detected mass-to-charge ratios of the various ion fragments that a trisulfide bond is present within the analyte ion 100. By way of example, it will be appreciated that the various fragments containing the first portion 101 of the analyte ion 100 would represent a first pair 101a/b of ions differing from one another by the mass of a sulfur atom (i.e., about 32 amu) due to their differential fragmentation about the central sulfur in the trisulfide linkage. In other words, the fragment 101a would exhibit a mass 32 amu greater than fragment 101b. Likewise, the various fragments containing the second portion 102 of the analyte ion 100 would represent a second pair 102a/b of ions also differing from one another by the mass of a sulfur atom (i.e., about 32 amu) due to their differential fragmentation about the central sulfur. In particular, the fragment 102a would exhibit a mass 32 amu greater than fragment 102b. Wherein the system is configured to identify one or more pairs of fragment ions from the MS/MS data differing in mass from one another by about 32 amu, the present teachings provide for the determination of the likely existence of a trisulfide bond within the population of analyte ions. In various aspects, the determination of fragment pairs 101a/b and 102a/b can be displayed to the user.

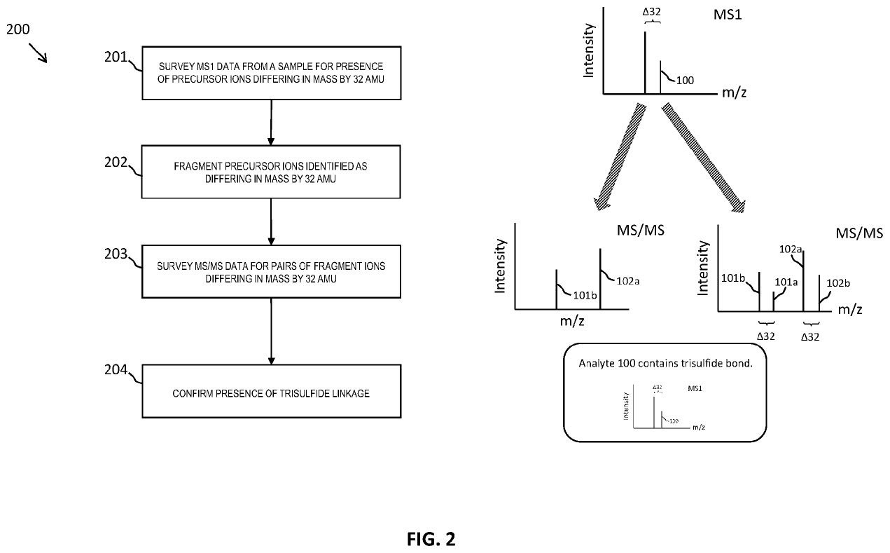

An exemplary method in accordance with various aspects of the present teachings in depicted in FIG. 2. Initially, in step 201, a MS scan can be performed to determine the m/z of ions within the population of analyte ions generated by the ion source. In certain aspects, it may be determined whether the population of ions contains “precursor” ions differing in mass by about 32 amu. For present purposes, these ions in the sample are referred to as “precursor” ions in the MS1 data as they are not subject to fragmentation in step 201. By way of example, with reference again to FIG. 1, if a sample contains both the depicted analyte ion 100 as well as the same ion but with a disulfide bridge, methods in accordance with certain aspects of the present teachings can identify in the MS1 data the potential presence of ions differing in only the mass corresponding to a sulfur atom. While this data suggests the presence of analytes containing disulfide bond and a modified analyte containing a trisulfide bond (e.g., resulting from a post translational modification in which a sulfur atom is inserted into a disulfide bridge of cysteine residues), the present teachings can provide further confirmation that a trisulfide linkage exists. By way of example, as shown in FIG. 2, the precursor ions separated by 32 amu identified in step 201 can then be subjected to MS/MS in step 202. For example, in a subsequent run, the precursor ions differing in 32 amu from step 201 can be mass filtered and subjected to fragmentation in a fragmentation device such that the fragment ions (also known as product ions) are mass analyzed to generate MS/MS data from each of the precursor ions identified in step 201.

In step 203, the MS/MS data can be analyzed to determine the presence of any pairs of fragment ions differing in mass by 32 amu. By way of example, if the precursor ion 100 containing the trisulfide linkage of FIG. 1 were subject to fragmentation in accordance with the present teachings, the MS/MS data would indicate two pairs of fragment ions, each separated from one another by 32 amu as schematically indicated in step 203. In this example, step 203 may include surveying the MS/MS data for a first pair of fragment ions 101a/b and a second pair of fragment ions 102a/b. It will be noted that the MS/MS data from a similar precursor ion to analyte 100 but lacking the trisulfide bond would likely not exhibit such pairs of fragment ions differing by 32 amu as both portions of the fragmented ion would only contain a single sulfur from one half of the disulfide bridge. Rather, the MS/MS data would likely indicate only the presence of fragment ion 101b and fragment ion 101a.

In step 204, the method can thereby confirm with high confidence that a portion of the sample contains ions exhibiting a trisulfide bond. In certain aspects, the confirmation of the presence of a trisulfide bond can be displayed to a user, for example, along with the identification of the precursor ion from which the MS/MS data was derived.

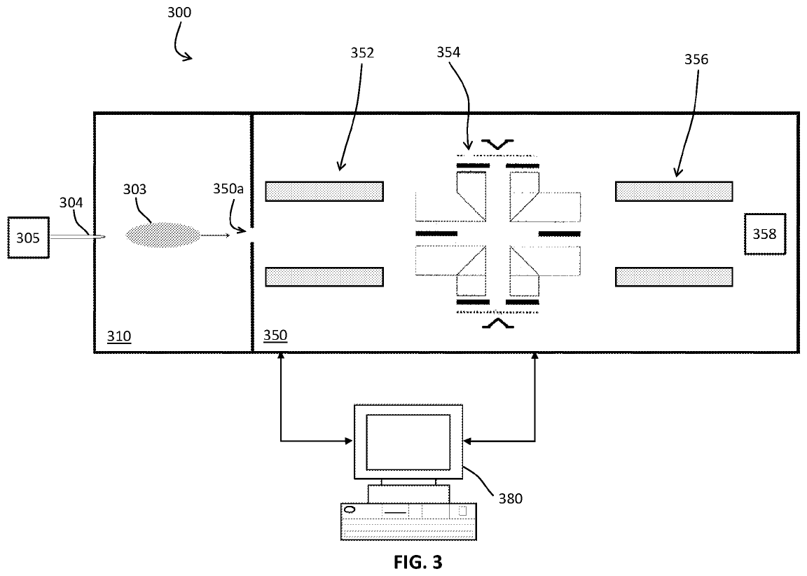

With reference now to FIG. 3, an embodiment of an exemplary system 300 for performing methods in accordance with various aspects of the applicant's teachings is schematically depicted. As shown, the system 300 generally comprises an ion source 304 configured to ionize sample ions for transmission to one or more downstream chambers (e.g., vacuum chamber 350) that may house a mass filter 352, a fragmentation device 354, a mass analyzer 356, and a detector 358 for ion processing in accordance with the present teachings. One or more power supplies (e.g., under the control of computer system 380) may be configured to apply various DC, AC, and/or RF signals to the various components of the system 300 for controlling the movement and processing of ions 303 within the system 300, as otherwise discussed herein.

The ions 303 transmitted into the vacuum chamber 350 can be generated by any known or hereafter developed ion source for generating ions and modified in accordance with the present teachings. Non-limiting examples of ion sources suitable for use with the present teachings include atmospheric pressure chemical ionization (APCI) sources, electrospray ionization (ESI) sources, continuous ion source, a pulsed ion source, an inductively coupled plasma (ICP) ion source, a matrix-assisted laser desorption/ionization (MALDI) ion source, a glow discharge ion source, an electron impact ion source, a chemical ionization source, or a photo-ionization ion source, among others.

In the example depicted in FIG. 3, the ion source 304 comprises an electrospray electrode, which can comprise a capillary fluidly coupled to a sample source 305 (e.g., through one or more conduits, channels, tubing, pipes, capillary tubes, etc.), and which terminates in an outlet end that at least partially extends into the ionization chamber 310 to discharge the liquid sample therein. As will be appreciated by a person skilled in the art in light of the present teachings, the outlet end of the electrospray electrode can atomize, aerosolize, nebulize, or otherwise discharge (e.g., spray with a nozzle) the sample into the ionization chamber 310 to form a sample plume comprising a plurality of micro-droplets generally directed toward (e.g., in the vicinity of) the mass spectrometer orifice 350a. As is known in the art, analytes contained within the micro-droplets can be ionized (i.e., charged) by the ion source 304, for example, as the sample plume is generated. By way of non-limiting example, the outlet end of the electrospray electrode can be made of a conductive material and electrically coupled to a pole of a voltage source (not shown), while the other pole of the voltage source can be grounded. Micro-droplets contained within the sample plume can thus be charged by the voltage applied to the outlet end such that as the desorption solvent within the droplets evaporates during desolvation in the ionization chamber 310 such bare charged analyte ions are released and drawn toward the orifice 350a. One or more power supplies can supply power to the ion source 304 with appropriate voltages for ionizing the analytes in either positive ion mode (analytes in the sample are protonated, generally forming cations to be analyzed) or negative ion mode (analytes in the sample are deprotonated, generally forming anions to be analyzed). Further, the ion source 304 can be nebulizer-assisted or non-nebulizer assisted. In some embodiments, ionization can also be promoted with the use of a heater (not shown), for example, to heat the ionization chamber so as to promote dissolution of the liquid discharged from the ion source.

Additionally, as shown in FIG. 3, the system 300 can include a sample source 305 configured to provide a sample to the ion source 304. The sample source 305 can be any suitable sample inlet system known in the art. By way of example, the ion source 305 can be configured to receive a fluid sample from a variety of sample sources, including a reservoir containing a fluid sample that is delivered to the sample source (e.g., pumped) or via an injection of a sample into a carrier liquid. In various example aspects, the sample source 305 may be a sample separation device utilizing techniques such as, but not limited to, liquid chromatography (LC), gas chromatography, or capillary electrophoresis. In some example aspects, the sample separation device may comprise an in-line liquid chromatography (LC) column, for example, that is configured to separate one or more compounds from a sample over time. In such aspects, the sample to be analyzed may be the eluent of the LC column, whose composition (and the analytes contained therein) may change over time, for example, based on binding affinity and/or the elution gradient applied to the LC column.

Although ions 303 are depicted in FIG. 3 as exiting the ionization chamber 310 via orifice 350a to enter the vacuum chamber 350, it will be appreciated that one or more intermediate vacuum chambers (not shown) may be disposed between the ionization chamber 310 and the vacuum chamber 350. Such intermediate vacuum chambers may be maintained at elevated pressures greater than the high vacuum chamber 350 within which the mass analyzers are disposed, and may contain one or more ion guides (e.g., quadrupoles) and/or ion optical elements to provide collisional cooling and/or help form an ion beam prior to delivering ions into the vacuum chamber 350. By way of non-limiting example, the ionization chamber 310 may be maintained at atmospheric or substantially atmospheric pressure (e.g., about 760 Torr), while the vacuum chamber 350 may be maintained at a pressure less than about 1×10⁴ Torr or lower (e.g., about 5×10⁻⁵ Torr), though other pressures can be used.

Ions transmitted into the vacuum chamber 350 via orifice 350a can enter the mass filter 352 (also referred to herein as Q1). As will be appreciated by a person of skill in the art, the mass filter 352 can be operated as a conventional transmission RF/DC quadrupole mass filter that can be operated to select an ion of interest and/or a range of ions of interest. For example, the computer system 380 can cause suitable RF/DC voltages to be applied to the mass filter 352 so as to operate in a mass-resolving mode. As should be appreciated, taking the physical and electrical properties of mass filter 352 into account, parameters for an applied RF and DC voltage can be selected so that mass filter 352 establishes a transmission window of chosen m/z ratios, such that these ions can traverse Q1 largely unperturbed. Ions having m/z ratios falling outside the window, however, do not attain stable trajectories within the quadrupole and can be prevented from traversing the mass filter 352. It should be appreciated that this mode of operation is but one possible mode of operation for mass filter 352. For example, in some aspects, the mass filter 352 can be operated in a RF-only transmission mode in which a resolving DC voltage is not utilized such that substantially all ions of the ion beam pass through the mass filter 352 largely unperturbed (e.g., ions that are stable at and below Mathieu parameter q=0.908). Alternatively, one or more ion optical elements (not shown) between the mass filter 352 and the fragmentation device 354 can be maintained at a much higher offset potential than mass filter 352 such that mass filter 352 can be operated as an ion trap. In such a manner, the potential applied to the ion optical elements (not shown) can be selectively lowered (e.g., mass selectively scanned) such that ions trapped in mass filter 352 can be accelerated into fragmentation device 354, which could also be operated as an ion trap, for example.

Ions transmitted by the mass filter 352 enter into the adjacent fragmentation device 354, which in some implementations, can be effective to fragment ions therewithin. For example, when in MS/MS mode, the mass filter 352 can be operated to transmit to fragmentation device 354 precursor ions exhibiting a selected range of m/z for fragmentation into product ions within fragmentation device 354. In MS mode, however, a person skilled in the art will appreciate that the fragmentation device 354 can be operated such that ions received from the mass filter pass through the fragmentation device 54 largely unperturbed (e.g., without substantial fragmentation).

The fragment ions can be generated within the fragmentation device 354 using any fragmentation technique known in the art or hereafter developed. By way of non-limiting example, fragment ions can be generated via collision induced dissociation (CID), as is known in the art. For example when using CID-based techniques, the fragmentation device 354 can be disposed in a pressurized compartment and can be configured to operate as a collision cell at a pressure approximately in the range of from about 1 mTorr to about 10 mTorr, though other pressures can be used for this or for other purposes. A suitable collision gas (e.g., nitrogen, argon, helium, etc.) can be provided by way of a gas inlet (not shown) to thermalize and/or fragment ions within the ion beam.

In certain aspects, it has been found that electron activated dissociation (EAD) techniques such as electron capture dissociation (ECD) may be particularly effective at fragmenting the precursor ions at the trisulfide bond such that for each trisulfide linkage, two pairs of fragment ions are reliably generated from the dissociation. Thus, in some example aspects as depicted in FIG. 3, the fragmentation device 354 is an electron reaction device that is configured to generate fragment ions through EAD-based techniques. Known mechanisms for EAD can include, for example, electron transfer dissociation (ETD), electron capture dissociation (ECD) using electrons having kinetic energies of 0 to 3 eV, Hot ECD (electrons with kinetic energy of 5 to 10 eV), and high energy electron ionization dissociation (HEEID) (electrons with kinetic energy greater than 13 eV). These electron activated dissociations are considered to be complimentary to conventional collision induced or activated dissociations (CID or CAD) and have been incorporated in advanced MS devices. The usage of the term EAD in the present teachings hereinafter should be understood to encompass all forms of electron-related dissociation techniques, and is not limited to the usage of electrons within any specific degree of kinetic energy. Known examples of EAD-capable fragmentation devices suitable for use in accordance with various aspects of the present teachings are described, for example, in PCT Pub. No. 2014/191821 entitled “Inline Ion Reaction Device Cell and Method of Operation,” the teachings of which are incorporated herein in its entirety. In certain aspects, the EAD-based fragmentation device may utilize a beam of electrons transmitted in a transverse direction relative to the ions passing through the fragmentation device 356 to induce collisions and reactions. For example, the electrons can be generated by an electron source such as a tungsten or thoriated tungsten filament or other electron source such as a Y₂O₃ cathode. Inside the fragmentation device 356, the ions and electrons interact, which can cause a number of phenomena to occur resulting in the formation of fragment or product ions, which can then be extracted or ejected from the fragmentation device 356 together with potentially other unreacted ions. For additional teachings on electron activated dissociation, see U.S. Patent Pub. No. 20180005810 entitled “Electron Induced Dissociation Devices and Methods filed Dec. 21, 2015, and PCT App. No. PCT/IB2012/002621, entitled “Ion Extraction Method For Ion Trap Mass Spectrometry” filed on Dec. 6, 2012, each of which is also incorporated herein by reference in its entirety.

Ions that are transmitted by fragmentation device 154 can pass into the adjacent mass analyzer 356, which can be operated in a number of manners, for example, as a scanning RF/DC quadrupole, as a linear ion trap, or as a RF-only ion guide to allow the ions to pass therethrough unperturbed. Suitable mass analyzers 356 for use in accordance with the present teachings include a time-of-flight (TOF) device, a quadrupole, an ion trap, a linear ion trap, an orbitrap, a magnetic four-sector mass analyzer, a hybrid quadrupole time-of-flight (Q-TOF) mass analyzer, or a Fourier transform mass analyzer, all by way of non-limiting example. In some aspects, for example, mass analyzer 356 can be operated as an ion trap for trapping ions received from the fragmentation device 354, with the potentials applied to exit ion optical elements (not shown) being selectively lowered such that ions trapped within mass analyzer 156 can be transmitted in a mass-selective manner to detector 358, which generates ion detection signals in response to the incident ions.

The computer system 380, which is in communication with the detector 358, may receive and process the ion detection signals to generate a mass spectrum of ions, for example, indicating the amount of ions (e.g., intensity, count) of each m/z that were transmitted by the mass analyzer 356.

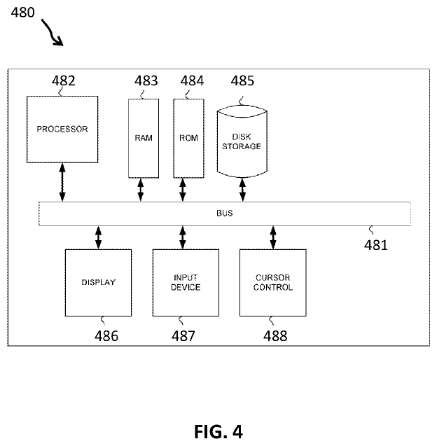

FIG. 4 is a block diagram that illustrates a computer system 480, upon which embodiments of the present teachings may be implemented. Computer system 480 includes a bus 481 or other communication mechanism for communicating information, and a processor 482 coupled with bus 481 for processing information. Computer system 480 also includes a memory 483, which can be a random access memory (RAM) or other dynamic storage device, coupled to bus 481 for storing instructions to be executed by processor 482. Memory 483 also may be used for storing temporary variables or other intermediate information during execution of instructions to be executed by processor 482. Computer system 480 further includes a read only memory (ROM) 484 or other static storage device coupled to bus 481 for storing static information and instructions for processor 482. A storage device 485, such as a magnetic disk or optical disk, is provided and coupled to bus 481 for storing information and instructions.

Computer system 480 may be coupled via bus 481 to a display 486, such as a cathode ray tube (CRT) or liquid crystal display (LCD), for displaying information to a computer user. An input device 487, including alphanumeric and other keys, is coupled to bus 481 for communicating information and command selections to processor 482. Another type of user input device is cursor control 488, such as a mouse, a trackball or cursor direction keys for communicating direction information and command selections to processor 482 and for controlling cursor movement on display 486. This input device typically has two degrees of freedom in two axes, a first axis (i.e., x) and a second axis (i.e., y), that allows the device to specify positions in a plane.

A computer system 480 can perform the present teachings. Consistent with certain implementations of the present teachings, results are provided by computer system 480 in response to processor 482 executing one or more sequences of one or more instructions contained in memory 483. Such instructions may be read into memory 483 from another computer-readable medium, such as storage device 485. Execution of the sequences of instructions contained in memory 483 causes processor 482 to perform the process described herein. Alternatively, hard-wired circuitry may be used in place of or in combination with software instructions to implement the present teachings. Thus, implementations of the present teachings are not limited to any specific combination of hardware circuitry and software.

For example, the present teachings may be performed by a system that includes one or more distinct software modules for perform a method for analyzing ions in accordance with various embodiments (e.g., a mass filter module, a fragmentation module, an analyzer module, a display module).

In various embodiments, computer system 480 can be connected to one or more other computer systems, like computer system 480, across a network to form a networked system. The network can include a private network or a public network such as the Internet. In the networked system, one or more computer systems can store and serve the data to other computer systems. The one or more computer systems that store and serve the data can be referred to as servers or the cloud, in a cloud computing scenario. The one or more computer systems can include one or more web servers, for example. The other computer systems that send and receive data to and from the servers or the cloud can be referred to as client or cloud devices, for example.

The term “computer-readable medium” as used herein refers to any media that participates in providing instructions to processor 482 for execution. Such a medium may take many forms, including but not limited to, non-volatile media, volatile media, and transmission media. Non-volatile media includes, for example, optical or magnetic disks, such as storage device 485. Volatile media includes dynamic memory, such as memory 483. Transmission media includes coaxial cables, copper wire, and fiber optics, including the wires that comprise bus 481.

Common forms of computer-readable media or computer program products include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer readable media may be involved in carrying one or more sequences of one or more instructions to processor 482 for execution. For example, the instructions may initially be carried on the magnetic disk of a remote computer. The remote computer can load the instructions into its dynamic memory and send the instructions over a telephone line using a modem. A modem local to computer system 480 can receive the data on the telephone line and use an infra-red transmitter to convert the data to an infra-red signal. An infra-red detector coupled to bus 481 can receive the data carried in the infra-red signal and place the data on bus 481. Bus 481 carries the data to memory 483, from which processor 482 retrieves and executes the instructions. The instructions received by memory 483 may optionally be stored on storage device 485 either before or after execution by processor 482.

The descriptions herein of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software, though the present teachings may be implemented as a combination of hardware and software or in hardware alone. The present teachings may be implemented with both object-oriented and non-object-oriented programming systems.

The section headings used herein are for organizational purposes only and are not to be construed as limiting. While the applicant's teachings are described in conjunction with various embodiments, it is not intended that the applicant's teachings be limited to such embodiments. On the contrary, the applicant's teachings encompass various alternatives, modifications, and equivalents, as will be appreciated by those of skill in the art.

What is claimed is:

1. A computer-implemented method of determining the presence of trisulfide bonds in a sample, comprising:
instructing, using a processor, a fragmentation device to generate a plurality of fragment ions from a population of analyte ions;
instructing, using the processor, a mass analyzer to generate data indicative of the m/z of the plurality of fragment ions; and
based on the data indicative of the m/z of the plurality of fragment ions, identifying at least a first pair of fragment ions, if any, differing in mass from one another by about 32 mass units using the processor.

2. The method of claim 1, wherein the fragmentation device is configured to generate the plurality of fragment ions using electron activated dissociation.

3. The method of claim 1, wherein the plurality of fragment ions are generated by electron capture dissociation.

4. The method claim 1, further comprising identifying a second pair of fragment ions differing in mass from one another by about 32 mass units using the processor, wherein the second pair of fragment ions differ in mass from the first pair of fragment ions.

5. The method of claim 1, further comprising instructing, using the processor, the mass analyzer to generate data indicative of the m/z of the population of analyte ions.

6. The method of claim 5, further comprising identifying a precursor ion corresponding to the first pair of fragment ions and a second pair of fragment ions identified based on the data indicative of the m/z of the population of analyte ions, wherein the second pair of fragment ions differ in mass from one another by about 32 mass units and wherein the second pair of fragment ions differ in mass from the first pair of fragment ions.

7. The method of claim 1, wherein the sample is subjected to liquid chromatography prior to being subject to fragmentation.

8. The method of claim 1, further comprising:
generating a mass spectrum, using the processor, based on the measured m/z of the plurality of fragment ions; and
using the processor, identifying at least two pairs of peaks in said mass spectrum, each pair corresponding to fragment ions differing in mass from one another by about 32 mass units.

9. The method of claim 1, wherein the population of analyte ions comprise polypeptides.

10. The method of claim 9, wherein the polypeptides comprise antibodies.

11. A method of determining the presence of trisulfide bonds in a sample, comprising:
performing electron activated dissociation on a population of analyte ions to generate a plurality of fragment ions;
identifying two pairs of fragment ions from the plurality of fragment ions, wherein fragment ions in each pair differ in mass from one another by about 32 mass units.

12. The method of claim 11, wherein the electron activated dissociation comprises electron capture dissociation.

13. The method of claim 11, further comprising mass analyzing the plurality of fragment ions to measure the m/z and intensity of the plurality of fragment ions.

14. The method of claim 11, further comprising mass analyzing the population of analyte ions to identify a precursor ion to the two pairs of fragment ions.

15. The method of claim 11, wherein the sample is subjected to liquid chromatography prior to being subjected to electron activated dissociation.

16. The method of claim 11, wherein the analyte ions comprise polypeptides.

17. The method of claim 16, wherein the polypeptides comprise antibodies.

18-26. (canceled)

27. A system for analyzing a sample comprising:
a tandem mass spectrometer comprising:
an ion filter,
at least one of a collision cell and an electron activated dissociation device, and
a mass analyzer,
a processor configured to:
instruct the tandem mass spectrometer to perform a MS1 scan of the sample by mass analyzing the sample,
receive from the tandem mass spectrometer an MS1 spectra of the sample,
identify at least one pair of peaks in the MS1 spectra that differ from one another in m/z by 32 units,
for each of said at least one pair of peaks, instructs the tandem mass spectrometer to isolate a precursor ion representative of each of the peaks in each pair of the at least one pair of peaks and perform an electron activated fragmentation on each precursor ion and to generate fragment ions and to mass analyze said fragment ions,
receive from the tandem mass spectrometer an MSMS spectra of said fragment ions,
survey the MSMS spectra for one or more fragment peaks pairs, wherein in each fragment peak pair, the m/z for each differs from the other by 32,
determines whether the fragment peak pairs correlates by determining whether said fragment peaks were derived from the precursor ion of any of the at least one pair of peaks in the MS1 spectra and if so, determining that a tri-sulfide linkage exists in said sample.
